(19) United States
(12) Patent Application Publication
(10) Pub. No.: US 2008/0124714 A1
(43) Pub. Date: May 29, 2008

(54) Method for Stabilizing Biological Samples for Nucleic Acid Analysis

(75) Inventors: Anthony P. Shuber, Mendon, MA (US); Duncan H. Whitney, Sudbury, MA (US)

Correspondence Address: GOODWIN PROCTER LLP;PATENT ADMINISTRATOR, EXCHANGE PLACE, BOSTON, MA, 02109-2881, US

(73) Assignee: Exact Sciences Corporation, Marlborough, MA (US)

(21) Appl. No.: 11/596,400

(22) PCT Filed: May 16, 2005
(86) PCT No.: PCT/US05/17046

§ 371 Date: Jan. 10, 2008

Related U.S. Application Data
(60) Provisional application No. 60/571,120, filed on May 14, 2004.

(51) Int. Cl. C12Q 1/68

(52) U.S. Cl. 435/6

(57) ABSTRACT

Disclosed is a method for preparing nucleic acid containing biological samples for a nucleic acid integrity assay and/or multiple mutation analysis by incubating the biological sample with a stabilization solution that includes a buffer, a chelating agent and a salt.

### RELATED APPLICATIONS

This application claims priority under 35 U.S.C. § 119(e) to U.S. Provisional Application Ser. No. 60/571,120, entitled “Method for Stabilizing a Biological Sample for a Nucleic Acid Integrity Assay” filed on May 14, 2004, the disclosure of which is herein incorporated by reference in its entirety.

### FIELD OF THE INVENTION

The invention relates generally to assays to detect nucleic acid markers indicative of cancer and other diseases and more particularly to preparing nucleic acid-containing biological samples for use in these assays.

### BACKGROUND OF THE INVENTION

Tissue and body fluid samples, including stools, contain shed cellular debris. In healthy patients, such debris is the result of apoptotic degradation as part of the normal cell cycle. Apoptosis reduces the integrity (intactness) of nucleic acids, proteins, and other cellular components in healthy individuals, so that only small fragments exist in the debris that results from the apoptotic process (e.g., exfoliated cellular debris).

In diseases in which cell cycle mechanisms are destroyed or impaired, cellular debris can include high-integrity cellular components, such as nucleic acids that have not been degraded by apoptosis. One class of disease in which cell cycle mechanisms are disrupted is cancer. An increased presence of high molecular weight nucleic acids in a biological sample therefore can reveal the presence of cancer in a patient from whom the biological sample was obtained. Disease detection assays known as nucleic acid integrity analysis assays have been developed that are based on the increased levels of non-degraded nucleic acid in a cancerous tissue or body fluid as compared to the level of non-degraded nucleic acid in a non-cancerous tissue or body fluid.

Nucleic acids in patient samples tend to degrade after they have been removed from the patient. This degradation can diminish the effectiveness of a nucleic acid integrity assay that scores a sample as diseased (e.g., cancerous) based on the presence of intact nucleic acids; if the sample is excessively degraded, a sample that is actually positive may appear to be negative.

### SUMMARY OF THE INVENTION

The invention is based in part on the discovery of a method for stabilizing nucleic acids in tissue and body fluid samples so as to facilitate analysis of the samples in a nucleic acid integrity assay. It has been unexpectedly found that contacting a patient sample with a stabilization solution stabilizes the DNA so that intact nucleic acids indicative of diseased cells are more effectively detected in a nucleic acid integrity assay.

Accordingly, in one aspect, the invention provides a method for preparing a nucleic acid sample for a nucleic acid integrity analysis assay. In one aspect, the invention provides a method for preparing a nucleic acid sample for a nucleic acid integrity analysis assay. The method includes providing a patient sample that includes shed cells or cellular debris and a nucleic acid and contacting the patient sample with a stabilization solution under conditions sufficient to stabilize the nucleic acid for nucleic acid integrity analysis. In one embodiment, the stabilization solution includes a buffer, a salt, and a chelating agent.

In some embodiments, the conditions are sufficient to detect at least a three-fold genomic equivalent (GE) increase in a nucleic acid integrity analysis of a patient sample having adenoma or cancer as compared to the GE detected in a nucleic acid integrity analysis of a sample from the patient that is not incubated with the stabilization solution. In one embodiment, the integrity analysis is performed by determining an amount of nucleic acid greater than about 200 bp in length using an assay that detects a nucleic acid (which can be a wild-type or mutant nucleic acid). The nucleic acid is present in a patient sample that includes shed cells or cellular debris. A patient is identified as having cancer or adenoma if the amount of nucleic acid is greater than an amount of nucleic acid expected to be present in a sample obtained from a patient who does not have cancer or adenoma.

The patient sample can be obtained from a patient that is, e.g., a vertebrate, including a mammal, a reptile, or an amphibian. A mammal can be, e.g., a human, a non-human primate (such as a gorilla or monkey, including a chimpanzee), a rodent (such as a mouse, rat, guinea pig, or gerbil) dog, cat, horse, pig, goat, sheep, or cow. The patient sample can be any body tissue or fluid that is suspected of containing DNA from a diseased cell (such as a precancerous or cancerous cell). In one embodiment, the patient sample is a stool sample.

In some embodiments, the patient sample can be obtained as part of a screen for, e.g., a disease or disease-associated condition that impairs, or could lead to impairment of, the proper function of the gastrointestinal system. Gastrointestinal diseases can include, e.g., those associated with the stomach, small intestine, and/or colon. The disease or condition can include cancers or precancerous conditions such as an adenoma. Other conditions include inflammatory bowel syndrome, inflammatory bowel disease, Crohn's disease, and others in which a genomic instability is thought to play a role.

In some embodiments, the patient sample is frozen and thawed prior to incubation with stabilization solution. In other embodiments, the patient sample is not frozen prior to incubation with the stabilization solution.

In general, the stabilization solution is added to the patient sample at a ratio of about 1 ml/gram of patient sample to about 20 ml/gram of patient sample. In some embodiments, the stabilization solution is provided at 1-15 ml/gram, 2-12 ml/gram, 3-11 ml/gram, or 4-7 ml/gram. However, higher or lower ratios may be used. When the patient sample is a stool sample, and the stabilization solution is 10 mM Tris-Cl, 1 mM EDTA, and 150 mM NaCL, a suitable ratio of stabilization solution to patient sample is 7 ml/gm.

In some embodiments, the patient sample and stabilization solution are incubated at about 4 to 28 degrees Centigrade. In some embodiments the temperature is 17 to 27 degrees Centigrade, e.g., about 20 to 25 degrees Centigrade. However, the sample and stabilization solution may be exposed to higher or lower temperatures (e.g., the sample and stabilization solution may be frozen). Also, a sample and buffer may be exposed to changing temperatures during transport and/or storage.

In various embodiments, the patient sample and stabilization solution are incubated at least 6 hours, e.g., at least 12 hours, at least 24 hours, or at least 36 hours.

In some embodiments, the buffer in the stabilization solution is 0.5 mM to 25 nM Tris, e.g., 5 mM to 15 mM Tris, 8 mM to 13 mM Tris or about 10 mM Tris. However, other buffers and/or concentrations may be used.

In some embodiments, the chelating agent in the stabilization solution is 0.01 to 2.5 mM EDTA, e.g., 0.75-1.25 mM EDTA, or 1 mM EDTA. However, other chelating agents and/or concentrations may be used.

In some embodiments, the salt in the stabilization solution is 75 mM to 225 mM NaCl, e.g., 100 mM to 175 mM NaCl, or 150 mM NaCl. However, other salts (e.g., KCl etc.) and/or concentrations may be used.

In some embodiments, the stabilization solution is provided at pH 7.0 to 9.0, e.g., pH 7.5 to 8.5, or pH 8.0. However, higher or lower pHs may be used.

In some embodiments, the method further includes determining in the incubated patient sample an amount of nucleic acid greater than about 200 bp in length using an assay that detects wild-type or mutant nucleic acid, and identifying the patient as having cancer or adenoma if the amount is greater than an amount of nucleic acid expected to be present in a sample obtained from a patient who does not have cancer or adenoma (e.g., more than about 1.5 fold, 2 fold, 3 fold, 4 fold, 5 fold, 10 fold, 50 fold, or more greater than an amount expected in a normal individual). In one aspect, a DNA integrity assay may include interrogating a sample for the presence of long DNA fragments (e.g., longer than 200 nucleotides, longer than 500 nucleotides, longer than 1,000 nucleotides, etc.) at two or more different loci. In one embodiment, a patient is identified as having cancer or adenoma if two or more loci (e.g., 3, 4, or more loci) are positive for the presence of abnormally high levels of long DNA.

In one embodiment, the invention provides a method for preparing a nucleic acid sample for a nucleic acid integrity and/or multiple mutation analysis assays for diagnosing a carcinoma or adenoma. The method may include providing a patient stool sample that includes shed cells or cellular debris and a nucleic acid, and incubating the patient sample with a stabilization solution under conditions sufficient to stabilize the nucleic acid for nucleic acid integrity and/or multiple mutation analysis. The stabilization solution may be about pH 7.5 to about pH 8.5 and may include 0.5 mM to 25 mM Tris, 0.01 to 2.5 mM EDTA and 100 mM to 200 mM NaCl. For example, the stabilization solution can be 10 mM Tris-Cl pH 8.0, 1 mM EDTA, and 150 mM NaCl. In one embodiment, the conditions are sufficient to detect at least a three-fold genomic equivalent (GE) increase in a nucleic acid integrity analysis of a patient sample having adenoma or cancer as compared to the GE detected in a nucleic acid integrity analysis of a sample from the patient that is not incubated with the stabilization solution.

Aspects of the invention may be used for transporting or storing biological samples after they are obtained and before they are processed for analysis. For example, methods of the invention may be used to stabilize nucleic acid in biological samples (e.g., stool samples) for about 12 hours, about 24 hours, about 36 hours, or longer (e.g., 4 days, 5, days, 6 days, 1 week, or longer), even in the absence of refrigeration or freezing.

Aspects of the invention may be particularly useful for detecting indicia of adenomas, early stage cancers, and/or other diseases that may be characterized by very low frequencies of mutant nucleic acid in a sample.

Aspects of the invention may be particularly useful for preserving biological samples for nucleic acid integrity assays or for multi-panel screens that include one or more nucleic acid integrity assays. Aspects of the invention may be particularly useful for detecting mutations in samples that contain very little human DNA (e.g., DIA negative samples). According to the invention, the amount of human DNA recovered from stool from different subjects may vary and it may be particularly important to preserve nucleic acids in samples that contain low amounts of human DNA.

Accordingly, aspects of the invention may be useful for screening a population to identify individuals with indicia of disease, and for avoiding or reducing the number of false negatives (subjects with a disease but who are identified as normal or healthy) in such screens. In one embodiment, average-risk individuals may screened for one or more indicia of a sporadic disease (e.g., adenoma, cancer, etc.). In one embodiment, high-risk individuals may be screened for a sporadic disease or a disease that may be inherited. In one embodiment, a screen may be performed on a population of individuals regardless of their risk profile.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a Table (Table 2) showing the results of DNA Integrity Assay (DIA) analysis performed on stool samples under different experimental conditions.

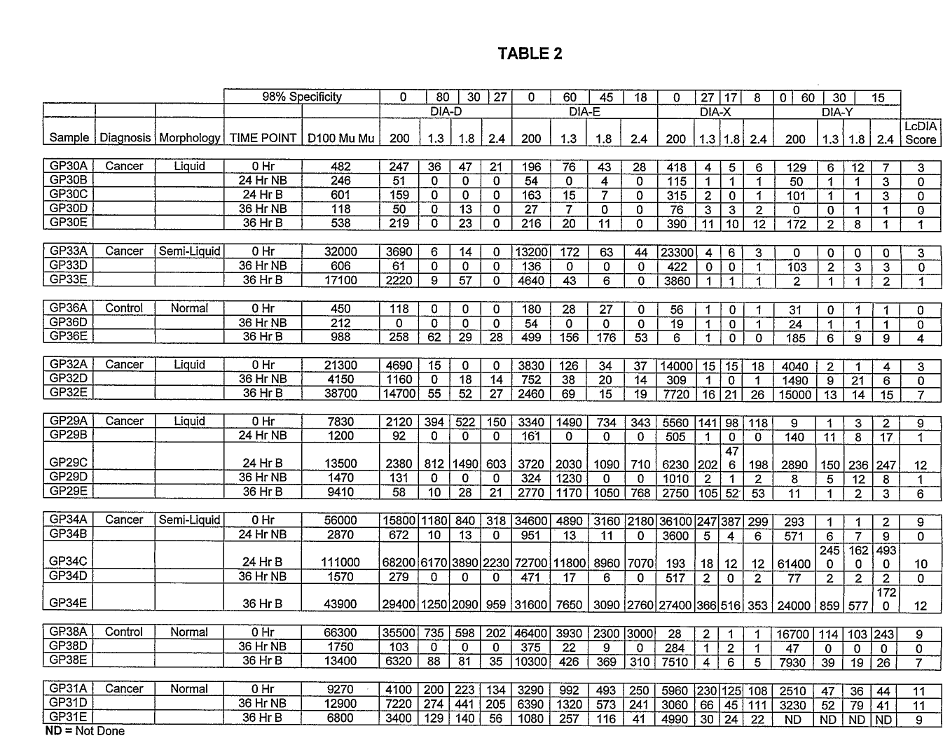

FIG. 2 is a Table (Table 5) showing the results of DNA Integrity Assay (DIA) analysis performed on stool samples under different experimental conditions.

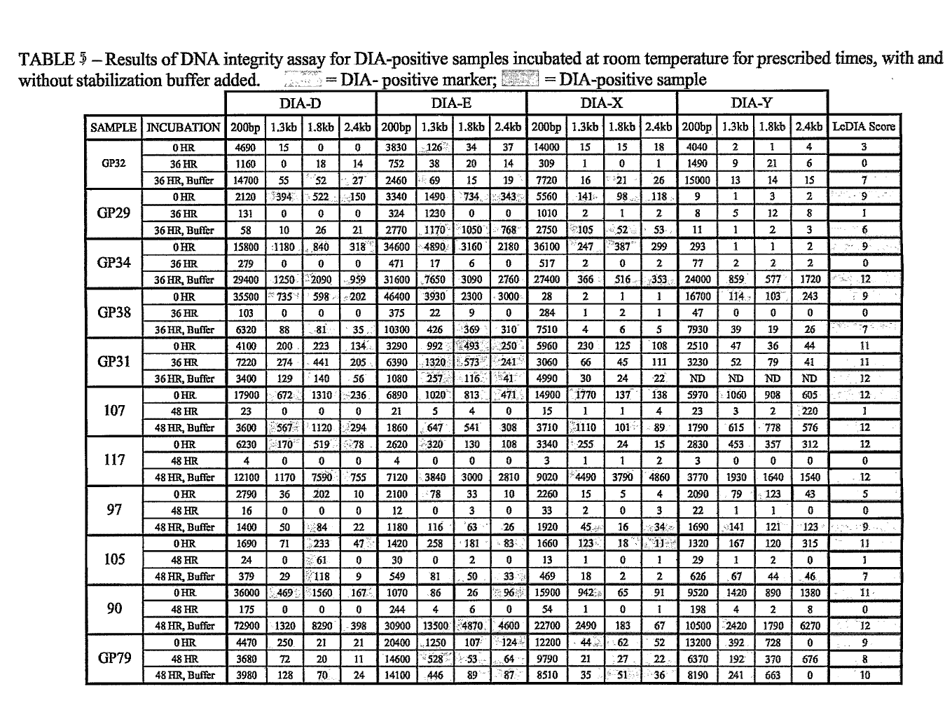

### DETAILED DESCRIPTION OF THE INVENTION

It has been unexpectedly found that a DNA integrity signal can be stabilized and/or enhanced by mixing, or incubating, a patient sample known to or suspect of containing DNA indicative of a disease with a stabilization solution prior to performing the DNA integrity assay. The stabilization solution typically includes one or more buffers, and/or chelating agents, and/or salts. Aspects of the invention are particularly useful for nucleic acid integrity assays. However, mutation detection (for example, in a multiple mutation assay) and hypermethylation analysis also can benefit from aspects of the invention.

According to the invention, an important challenge for colon cancer detection from stool is to preserve the integrity of human DNA in the hostile stool environment, in order to recover, amplify, and interrogate the DNA for known cancer related abnormalities. Nucleases that are active in stool have the potential to rapidly degrade DNA, including the minor human DNA component, and measures may be taken to minimize their negative impact. Typically, clinical samples are frozen as quickly as possible after collection. However, in order to use fecal DNA tests in population screens, it should be expected that there will be some variability in the time between sample collection and shipping to testing labs, and furthermore, some variability in the temperature at which stool samples are transported. In order to eliminate any variables in sample handling that might have an impact on assay performance we have run controlled sample incubation experiments and looked at how different markers in a multi-target assay are affected.

Markers may be chosen that yield an acceptable clinical sensitivity for the intended application such as screening a population for indicia of a disease. In addition, for stool sample analysis, mutation detection methods should offer sufficient analytical sensitivity since the human DNA recovered from stool is highly heterogeneous. Normal cells are sloughed into the colonic lumen along with the mutant cells. Therefore, in one embodiment, analytical methods should detect as little as 1% (or less) mutant DNA in the presence of excess wild-type DNA. Also, certain sample preparation methodologies may be used for maximum recovery of human DNA from samples. The vast majority of DNA recovered from stool often is bacterial in origin, with the human DNA component representing only a small minority. Certain purification methodologies can efficiently select for the rare human component, and since the mutant copies (when they exist) represent only a small percentage of the total human DNA from stool it may be important to maximize the recovery of human DNA in order to maximize the probability of amplifying mutant copies in the PCR reactions. In one embodiment, gel electrophoresis methods for capturing human DNA may be used. However, according to the invention, it may be particularly important to preserve sample DNA for purification, especially when looking for early indicia of diseases (e.g., indicia of adenomas or early stage cancers that may be present in less than about 1%, or about 0.1% or less of human genomes isolated from a stool sample). A common method to insure that DNA remains stable is to freeze stool samples as quickly as possible after collection, or to receive samples in centralized testing labs as quickly as possible. However, in order to provide the option of decentralized sample analysis and still retain maximum sample integrity, it is desirable to use a more robust and standardized sample handling method.

In one aspect, the invention provides methods for stabilizing biological samples (e.g., stool samples) by adding a stabilization solution to a sample as soon as possible after the sample is obtained. Methods of the invention do not require refrigeration or freezing. Aspects of the invention are based, in part, on the surprising discovery that nucleic acids in certain biological samples are stable at room temperature for hours, and even days (e.g., 1 day, 2 days, 3 days, or longer). However, in certain embodiments, samples with stabilization solution may be refrigerated or frozen. Aspects of the invention are particularly useful for preserving samples for nucleic acid integrity analysis. However, methods of the invention may be used to preserve samples for other assays including mutation detection and/or hypermethylation assays. In certain embodiments, methods of the invention are used to preserve a sample for analysis using a nucleic acid integrity assay along with a mutation detection assay (e.g., a multiple mutation panel assay), a hypermethylation assay, or both.

Nucleic acid integrity assays are known in the art and are described in, e.g., US Patent Application No. 20040043467, US Patent Application No. 20040014104, U.S. Pat. No. 6,143,529, and Boynton et al., Clin. Chem. 49:1058-65, 2003. Nucleic acid integrity assays are based on higher levels of intact nucleic acid that appear in debris from cells that lyse non-apoptotically. Healthy patient generally produces cellular debris through normal apoptotic degradation, resulting in relatively small fragments of cellular components in tissue and body fluid samples, especially luminal samples. Patients having a disease generally produce cells and cellular debris, a proportion of which has avoided normal cell cycle regulation, resulting in relatively large cellular components. As a result, the disease status of a patient is determined by analysis of patient cellular components produced in specimens obtained from the patient. The presence of such fragments is a general diagnostic screen for disease.

Nucleic acids in patient samples tend to degrade after they have been removed from the patient. This degradation can diminish the effectiveness of a nucleic acid integrity assay that scores a sample as diseased (e.g., cancerous) based on the presence of intact nucleic acids; if the sample is excessively degraded, a sample that is actually positive may appear to be negative. While not wishing to be bound by theory, it is postulated that the stabilization buffer of the invention inhibits the nucleases that degrade the nucleic acids present in the diseased patient samples.

In some aspects of the invention, the addition of a stabilization solution to a biological sample may be used to preserve nucleic acid molecules containing one or more mutations that may be detected in a multiple mutation analysis (e.g., an analysis that involves interrogating a sample for the presence of a mutation at one or more loci, for example at about 2, about 3, about 4, about 5, about 10, about 15, about 20, about 25, or more loci). In some embodiments, the addition of a stabilization solution may be used to preserve nucleic acid for a methylation specific analysis to detect the presence of hyper-methylated nucleic acid molecules at one or more loci that may be indicative of cancer, adenoma, or other disease. In some embodiments, the addition of a stabilization solution may be used to preserve nucleic acid for a combination of a nucleic acid integrity assay and/or a multiple mutation analysis and/or a methylation detection assay. Assays may be performed under conditions to detect a small amount of mutant nucleic acid in a heterogeneous sample containing an excess of non-mutant nucleic acid (e.g., where the mutant nucleic acid represents less than 10%, less than 5%, less than 1%, or about 0.1% or less of the nucleic acid at a particular locus). In some embodiments, a digital assay may be performed on the preserved nucleic acid in order to detect rare genetic events. In some aspects, stabilization methods of the invention may preserve more than 80%, 85%, 90%, 91%, 92%, 93%, 94%, 95%, 96%, 97%, 98%, 99% or more of the nucleic acids indicative of a disease (e.g., long nucleic acid fragments, nucleic acid molecules containing one or more specific mutations, and/or hyper-methylated nucleic acid molecules).

In general any body organ, tissue, or fluid known to or suspected of containing nucleic acid that can be characterized in a nucleic acid integrity analysis, multiple mutation assay, or methylation study may be used. Suitable patient samples include those likely to contain sloughed cellular debris. Such specimens include, but are not limited to, stool, blood serum or plasma, sputum, pus, colostrum, and others. In diseases, such as cancer, in which genomic instabilities or abnormalities have interfered with normal cell cycle regulation, specimens such as those identified above contain relatively intact fragments of cellular components.

The stabilization solution can be applied to a biological sample that is isolated directly from a patient, i.e., a freshly isolated biological sample. Alternatively, the method can be used on a biological sample that has been frozen (e.g., at −20° C. or −80° C.).

In aspects of the invention, stabilization solution may be added to a biological sample at any suitable ratio of sample to buffer. Ratios may be determined as a weight to volume (w/v) ratio. In some embodiments, the ratio may be about 1:1, about 1:2, about 1:3, about 1:4, about 1:5, about 1:6, about 1:7, about 1:8, about 1:9 or about 1:10 (w/v) sample to stabilization solution. However, higher or lower ratios may be used.

In aspects of the invention, a biological sample may be weighed before a buffer is added. In some embodiments, a sample (e.g., a stool sample) may weigh about 5 g, about 10 g, about 15 g, about 20 g, about 25 g, about 30 g, about 35 g, about 40 g, about 45 g, about 50 g, about 55 g, about 60 g, about 65 g, about 70 g, about 75 g, about 80 g, about 85 g, about 90 g, about 95 g, about 100 g, or more. It should be appreciated that a large amount of sample (e.g., over 25 g, 30 g, 50 g, 100 g, or more of stool) may be required in order to detect conditions such as adenomas or early stage cancers where a very small amount of mutant or abnormal nucleic acid may be present in a sample. According to the invention, it may be particularly important to immediately stabilize biological samples with a stabilization solution when the samples are to be interrogated for indicia of adenoma or early stage cancer. However, it also may be useful to stabilize biological samples for detecting indicia of later stage diseases.

In general, a stabilization solution may include one or more buffers and/or one or more chelating agents and/or one or more salts, or any combination of two or more thereof. The choices of buffer, chelating agent, and salt can be determined by the artisan. The suitability of a particular stabilization solution can be determined by comparing a nucleic acid integrity assay on samples that have been incubated with the stabilization solution to a parallel biological sample that has not been incubated with the stabilization solution. A suitable stabilization solution is a solution that shows a significant average fold increase in genome equivalents (GE) in a nucleic acid integrity assay compared to a GE determination made on parallel samples that have not been treated with the stabilization solution. Methods of calculating genome equivalents (GE) are known in the art (see, e.g., e.g., US Patent Application No. 20040043467, US Patent Application No. 20040014104, U.S. Pat. No. 6,143,529, and Boynton et al., Clin. Chem. 49:1058-65, 2003) and are illustrated in the Examples.

The temperature and pH optimum can also be determined empirically and optimized according to the combination of buffer, chelating agent and salt in the stabilization solution. While room temperature has been found to be a suitable temperature for incubating the patient sample and the stabilization solution, higher or lower temperatures (e.g., 4° C. to 16° C. or 25° C. to about 37° C.) can also be used, provided they do not undermine the effectiveness of the stabilization solution. The mixed patient sample and stabilization solution is preferably subjected to a minimum of agitation. However, according to the invention, the addition of a stabilization solution with little or no agitation is surprisingly effective at preserving nucleic acids for subsequent analysis.

In one aspect of the invention, a stabilization solution may be particularly useful when samples are not refrigerated or frozen or when there is a risk that a sample may not be maintained at a sufficiently low temperature to preserve indicia of disease. For example, a stabilization solution may be particularly useful if a sample is obtained at a remote location and mailed or delivered to a testing center. However, stabilization solutions also may be useful to preserve samples that are being processed on-site at a medical center.

### Buffers

Suitable buffers include, e.g. tris(hydroxymethyl)aminomethane, sodium phosphate, sodium acetate, MOPS, and other buffering agents as long as a buffer has the capacity to resist a 0.1 to 1 molar tris(hydroxymethyl)aminomethane or 0.1 to 1 molar phosphate ion. A combination of buffering agents can be used, so long as the solution has the required buffering capacity. Methods for determining the buffering capacity of a solution are well known in the art.

The comparison of buffering capacity is preferably carried out in the presence of the salt and chelating agent to be used in the stabilization solution, at the salt concentration to be used, and with the solutions being compared at about the same temperature, preferably at a temperature within the range of about 15° C. to about 25° C.

### Chelating Agents

Table 1 provides a representative list of chelating agents that can be used in the stabilization solution. The list provided in Table 1 is not meant to be exhaustive. In some embodiments, chelating agents are those which bind trace metal ions with a binding constant ranging from 101 to 10100; in some embodiments, chelating agents bind trace metal ions with a binding constant ranging from 1010 to 1080; in some embodiments, the chelators bind trace metal ions with a binding constant ranging from 1015 to 1060.

TABLE 1
Examples of Chelating Agents
CHELATORS

| ABBREVIATION | FULL NAME |
|---|---|
| EDTA free acid | Ethylenediamine-N,N,N′,N′,-tetraacetic acid |
| EDTA 2Na | Ethylenediamine-N,N,N′,N′,-tetraacetic acid, disodium salt, dihydrate |
| EDTA 3Na | Ethylenediamine-N,N,N′,N′,-tetraacetic acid, trisodium salt, trihydrate |
| EDTA 4Na | Ethylenediamine-N,N,N′,N′-tetraacetic acid, Tetrasodium salt, tetrahydrate |
| EDTA 2K | Ethylenefisminr-N,N,N′,N′-tetraacetic acid, dipotassium salt, dihydrate |
| | Ethylenefisminr-N,N,N′,N′-tetraacetic acid, dipotassium salt, dihydrate |
| EDTA 2Li | Ethylenediamine-N,N,N′,N′-tetraacetic acid, dilithium salt, monhydrate |
| EDTA 2NH₄ | Ethylenediamine-N,N,N′,N′-tetraacetic acid, diammonium salt |
| EDTA 3K | Ethylenediamine-N,N,N′,N′-tetraacetic acid, Tripotassium salt, dihydrate |
| Ba(ll)-EDTA | Ethylenediamine-N,N,N′,N′-tetraacetic acid, barium chelate |
| Ca(II)-EDTA | Ethylenediamine-N,N,N′,N′-tetraacetic acid, calcium chelate |
| Ce(III)-EDTA | Ethylenediamine-N,N,N′,N′-tetraacetic acid, cerium chelate |
| Co(II)-EDTA | Ethylenediamine-N,N,N′,N′-tetraacetic acid, cobalt chelate |
| Cu(11)-EDTA | Ethylenediamine-N,N,N′,N′-tetraacetic acid, copper chelate |
| Dy(Ill)-EDTA | Ethylenediamine-N,N,N′,N′-tetraacetic acid, dysprosium chelate |
| Eu(Ill)-EDTA | Ethylenediamine-N,N,N′,N′-tetraacetic acid, europium chelate |
| Fe(III)-EDTA | Ethylenediamine-N,N,N′,N′-tetraacetic acid, iron chelate |
| In(III)-EDTA | Ethylenediamine-N,N,N′,N′-tetraacetic acid, indium chelate |
| La(l1l)-EDTA | Ethylenediamine-N,N,N′,N′-tetraacetic acid, lanthanum chelate |
| Mg(II)-EDTA | Ethylenediamine-N,N,N′,N′-tetraacetic acid, magnesium chelate |
| Mn([1)-EDTA | Ethylenediamine-N,N,N′,N′-tetraacetic acid, manganese chelate |
| Ni(II)-EDTA | Ethylenediamine-N,N,N′,N′-tetraacetic acid, nickel chelate |
| Sm(lIl)-EDTA | Ethylenediamine-N,N,N′,N′-tetraacetic acid, samarium chelate |
| Sr(ll)-EDTA | Ethylenediamine-N,N,N′,N′-tetraacetic acid, strontium chelate |
| Zn(Il)-EDTA | Ethylenediamine-N,N,N′,N′-tetraacetic acid, zinc chelate |
| CyDTA | trans-1,2-Diaminocyclohexane-N,N,N′,N′-tetraaceticacid, monohydrate |
| DHEG | N,N-Bis(2-hydroxyethyl)glycine |
| DTPA-OH | 1,3-Diamino-2-hydroxypropane-N,N,N′,N′-tetraacetic acid |
| DTPA | 1,3-Di aminopropane-N,N,N′,N′-tetraacetic acid |
| EDDA | Ethylenediamine-N,N′-diacetic acid |
| EDDP | Ethylenediamine-N,N′-dipropionic acid dihydrochioride |
| EDDPO | Ethylenediamine-N,N′-bis(methylenephosphonic Acid), hemihydrate |
| EDTA-OH | N-(2-Hydroxyethyl)ethylenediamine-N,N′,N′-triacetic acid |
| EDTPO | Ethylenediamine-N,N,N′,N′-tetrakis(methylenephosponic acid) |
| EGTA | O,O′-bis(2-aminoethyl)ethyleneglycol-N,N,N′,N′-tetraacetic acid |
| HBED | N,N-bis(2-hydroxybenzyl)ethylenediamine-N,N-diacetic acid |
| HDTA | 1,6-Hexamethylenediamine-N,N,N′,N′-tetraacetic acid |
| HIDA | N-(2-Hydroxyethyl)irninodiacetic acid |
| IDA | Iminodiacetic acid |
| Methyl-EDTA | 1,2-Diaminopropane-N,N,N′,N′-tetraacetic acid |
| NTA | Nitrilotriacetic acid |
| NTP | Nitrilotripropionic acid |
| NTPO | Nitrilotris(methylenephosphoric acid), trisodium salt |
| O-Bistren | 7,19,30-Trioxa-1,4,10,13,16,22,27,33-octaabicyclo [11,11,11] pentatriacontane, |
| TTHA | Triethylenetetramine-N,N,N′,N″,N′′′,N′′′-hexaacetic acid |

### Salts

Candidate salts include, e.g, Nal, NaBr, NaCl, LiCl, KCl, KI, KBr, CsCl, GNHCl and GNSCN. In some embodiments, the salt is chaotropic and has an anion such as perchlorate, iodide, thiocyanate, acetate, trichloroacetate, hexafluorosilicate, tetrafluoroborate and the like. Cations for a chaotropic salt can include, e.g., the elements lithium, sodium, potassium, cesium, rubidium, guanidine and the like. More than one salt can be present in the buffered aqueous salt solution.

Patient samples that have been treated with a stabilization solution can be subject to the nucleic acid integrity assay. For example the treated sample can be used in method that includes determining an amount of nucleic acid greater than about 200 bp in length using an assay that detects wild-type or mutant nucleic acid, wherein said nucleic acid is present in a patient sample comprising shed cells or cellular debris; and identifying the patient as having cancer or adenoma if said amount is greater than an amount of nucleic acid expected to be present in a sample obtained from a patient who does not have cancer or adenoma.

### Adenomas

According to aspects of the invention, stabilization solutions may be particularly useful for preserving nucleic acids to detect one or more indicia of adenomas. Adenomas may be early indicia of cancer, for example colon cancer. Not all adenomas become cancers. However, many cancers (e.g., carcinomas such as colorectal carcinomas) are thought to develop from adenomas. Indeed, a majority of colon cancers are thought to develop from adenomas. Therefore, detecting adenomas is particularly useful for identifying early signs or risks of colorectal cancer (e.g., cancerous and precancerous lesions or growths in the colon).

Adenomas may be invasive adenocarcinomas, significant adenomas, and low potential polyps. Invasive adenocarcinomas may be, for example, adenocarcinomas at different TNM stages (e.g., TNM stages 1, 2, 3, or 4). Significant adenomas may be, for example, carcinomas in-situ/high-grade dysplasias (CIS/HGD) having a diameter of greater than 1 cm, about 1 cm, less than 1 cm, or of unknown size; vilous adenomas having a diameter of greater than 1 cm, about 1 cm, less than 1 cm, or of unknown size; tubulovillous adenomas having a diameter of greater than 1 cm, about 1 cm, less than 1 cm, or of unknown size, and low-grade dysplasias (LGD) with a diameter of greater than or equal to 1 cm. Low potential polyps may be, for example advanced polyps, and adenoma low-grade dysplasias (LGD) with an unknown diameter or a diameter of less than 1 cm.

According to aspects of the invention, adenomas can be detected at different positions in the colon and rectum (including the right and left colon and the transverse colon).

In one embodiment, the following panel may be used to detect adenomas with greater than 60% sensitivity: assays may be performed to detect one or more genetic abnormalities from a multiple mutation panel of genetic abnormalities at 22 loci including KRas mutations in codon 12 (K12p.1, K12p.2) and codon 13 (K13p.2); mutations in APC codons 1309 (deletions), 1306 (mutations at position 1), 1312 (mutations at position 1), 1367 (mutations at position 1), 1378 (mutations at position 1), 1379 (mutations at position 1), 1450 (mutations at position 1), 1465 (deletions), 876 (mutations at position 1) and 1554 (insertions); mutations in P53 codons 175p.2, 245p.1, 245p.2, 248p.1, 248p.2, 273p.1, 273p.2 and 282p.1; and deletions at the BAT-26 locus. Mutations at these loci can be detected using primer extension assays (including single base extension assays and assays designed to detect micro-satellite instability such as BAT-26 deletions) or other assays that are useful to detect one or more of these genetic abnormalities.

In another embodiment, the following panel may be used to detect adenomas with greater than 60% sensitivity: assays are performed to detect hypermethylation at one or both of the HLTF and V29 loci. Hypermethylation at these loci can be detected using methylation specific primer analysis (e.g., MSP amplification) or other assays that are useful to detect hypermethylation at one or more of these genetic loci.

In one embodiment, scanning for one or more mutations at the APC-MCR may detect adenomas with greater than 74% sensitivity.

In one embodiment, the following panel may be used to detect adenomas with greater than 90% sensitivity: scanning for one or more mutations in the APC-MCR locus, exon 9 of the PIK3CA locus, exon 20 of the PIK3CA locus, B-catenin (e.g., exon 5), or a mutation in BRAF that results in a V599E amino acid change. Scanning as described herein can be used to detect one or more mutations in the APC-MCR locus, exon 9 of the PIK3CA locus, or exon 20 of the PIK3CA locus. Mutations at the BRAF locus can be detected via primer extension or other appropriate methodology.

In one embodiment, a combination of all of the above loci may be used to detect adenomas with a greater than 95% sensitivity (e.g., greater than 98% sensitivity).

Any of the assays described herein may be performed in a digital format wherein a sample is diluted into aliquots wherein each aliquot contains on average between 1 and 20 target molecules of DNA for analysis (e.g., between 1 and 10 molecules, between 1 and 5 molecules, etc.).

The invention will be further illustrated in the following non-limiting examples.

### EXAMPLES

### Example 1

### Materials and Methods

### Sample Collection and Incubation

Samples were collected from known CRC patients as well as patients without cancer by a separate organization (Indivumed GmbH; Hamburg, Germany) that also managed all patient informed consent and compliance with human subject guidelines. All stool samples were frozen within 1 hour of defecation, and shipped to EXACT Sciences on dry ice (−78° C.). Once received, samples were subjected to prescribed room temperature incubation times as described below. Prior to the start of the incubation time-course stool samples were thawed and one aliquot was processed to recover DNA immediately (to). The DNA from the to-aliquot for all samples was analyzed and served as an incubation control. The remainder of the stool was left to incubate at room temperature. At prescribed time points aliquots were removed from the stool, human DNA recovered and analyzed in similar manner to the controls.

All aliquots were standardized by weight (30 g). An experiment was designed to measure the effect of incubation time on DNA integrity, as well as the quantity of recoverable DNA. The experiments also included an addition of stabilization buffer to stool aliquots. Stabilization buffer consisted of 0.5M Tris, 0.15M EDTA, and 10 mM NaCl (pH 9.0). In these experiments aliquots were stored at room temperature for 36 or 48 hours, with and without buffer added. In the case of buffer addition, the buffer was simply added to the stool aliquot, in a plastic container with a lid, but no effort was made to homogenize the sample. At the prescribed time period, the aliquots with and without buffer were processed to recover human DNA, and then analyzed by the DIA assay. An additional set of experiments was conducted to study the effect of incubation time on specific gene mutations. In this experiment one set of samples (6 samples) was incubated for 36, 48, or 72 hours without any stabilization buffer added. Another set of samples (5 samples) was incubated for 36 or 48 hours, with and without buffer added. After the prescribed incubation time all samples were processed to recover and purify human DNA, and analyze the DNA for gene mutations as described below.

### Recovery of DNA from Stool

The sample preparation methodology used to recover DNA from stool was previously reported (Ahlquist D A, Skoletsky J E, Boynton K A, et al. Colorectal cancer screening by detection of altered human DNA in stool: Feasibility of a multi-target assay panel. Gastroenterology 2000; 119:1219-1227; Whitney D, Skoletsky J, Moore K, et al. Enhanced Retrieval of DNA from Human Fecal Samples Results in Improved Performance of Colorectal Cancer Screening Assay. J Mol. Diagn. 2004; 6 (4), 386-395). Stool aliquots were weighed and combined with Exact buffer A (1:7 (w/v) ratio), and homogenized on an Exactor (Exact Sciences). After homogenization, a 4-g stool equivalent (−32 mls) of each sample was centrifuged to remove all particulate matter. The supernatants were then treated with 20 ul TE buffer (0.01 mol/L Tris [pH 7.4] and 0.001 mol/L EDTA) containing RNase A (2.5 mg/mL), and incubated at 37° C. for 1 hour. Total nucleic acid was then precipitated (first adding 1/10 volume 3 mol/L NaAc, then an equal-volume of isopropanol). Genomic DNA was pelleted by centrifugation, the supernatant removed, and the DNA resuspended in TE.

### Human DNA Purification

Target human DNA fragments were purified from total nucleic acid preparations using a newly developed DNA affinity electrophoresis purification methodology. This method has recently been described in detail 9. In brief, human DNA can be separated from the excess bacterial DNA by hybridization of the target sequences to complementary, covalently-bound oligonucleotide capture probes in acrylamide gels membranes. Crude human DNA preparations (2400 ul) were mixed with 960 ul formamide (Sigma), 385 ul 10×TBE, and filtered through a 0.8 um syringe filter (Nalgene, Rochester, N.Y.), then denatured (heated at 95C for 10 min, then cooled in ice for 5 min). The sample mix was loaded on top of the capture membrane, and electrodes above and below the capture layer were applied. Samples were electrophoresed (15V, 16 h) using TBE in the reservoirs above and below the capture layer. After electrophoretic capture the remaining solution was removed from the tubes, and the tube array was separated from the capture plate. The capture membranes were then washed and 40 ul of 100 mM NaOH was added to the top of the capture membrane and incubated for 15 min. The capture plate placed on top of a custom molded 48-well DNA collection plate and centrifuged briefly (1900×g) to recover the eluted DNA. Then 8 ul of neutralization buffer (500 mM HCL+0.1×TE) was added to each well of the collection plate and mixed.

### Quantification of Recovered Human DNA by TaqMan Analysis

TaqMan analysis was performed on an I-Cycler (BioRad) with primers against a 200-bp region of the APC gene. A probe labeled with 6-carboxyfluorescein (FAM) and 6-carboxytetramethylrhodamine (TAMRA) was used to detect PCR product. Amplification reactions consisted of captured human stool DNA mixed with 10×PCR buffer, LATaq enzyme (Takara), 1×PCR primers (5 μM), and 1×TaqMan probe (2 μM; Biosearch Technologies). We used 5 μl of captured DNA in the PCR reactions. TaqMan reactions were performed with the same program as described above (DIA).

### Sequence-Specific Amplification

Polymerase chain reaction (PCR) amplifications (50 μL) were performed on MJ Research Tetrad Cyclers (Watertown, Mass.) using 10 μL of purified DNA, 10×PCR buffer (Takara Bio Inc; Madison, Wis.), 0.2 mmol/L dNTPs (Promega, Madison, Wis.), 0.5 μmol/L sequence-specific primers (Midland Certified Reagent Co., Midland, Tex.), and 2.5 U LATaq DNA polymerase (Takara). All amplification reactions were performed under identical thermocycler conditions: 94° C. for 5 minutes, and 40 cycles consisting of 94° C. (1 min.), 60° C. (1 min.), and 72° C. (1 min.), with a final extension of 5 minutes at 72° C. Thirteen separate PCR reactions were run per sample. For analysis of each of the PCR products, 80, of each amplification reaction was loaded and electrophoresed on a 4% ethidium bromide-stained NuSieve 3:1 agarose gel (FMC, Rockland, Me.) and visualized with a Stratagene EagleEye II (Stratagene, La Jolla, Calif.) still image system.

The multi-target assay was designed to have 13 separate PCR reactions in the multiple mutation (MuMu) panel, and 16 PCR reactions in the DIA portion of the assay.

### Mutation Panel Analysis

The presence or absence of point mutations or Bat-26-associated deletions was determined by using modified solid-phase single-base extension (SBE) reactions. Point mutation targets included; codons K12p1, K12p2, and K13p2 on the K-ras gene; codons 876, 1306, 1309, 1312, 1367p1, 1378p1, 1379, 1450p1, 1465 and 1554 on the APC gene; and codons 175p2, 245p1, 245p2, 248p1, 248p2, 273p1, 2'73p2, and 282p1 on the p53 gene. Including the Bat-26 deletion marker, the panel consisted of 22 markers in total. For all gene targets, separate wild-type and mutant specific reactions were performed. Details of the reactions and analysis using capillary electrophoresis have been previously described (Whitney D, Skoletsky J, Moore K, et al. Enhanced Retrieval of DNA from Human Fecal Samples Results in Improved Performance of Colorectal Cancer Screening Assay. J Mol. Diagn. 2004; 6 (4), 386-395).

### DNA Integrity Assay (DIA)

The DIA assay has been previously described in detail. More recently this assay has been converted to a real-time PCR methodology. Three unique PCR reactions (in duplicate) per loci were run on I-Cycler instruments (BioRad; Hercules, Calif.). The strategy was to capture locus specific segments and perform small (−100 bp) PCR amplifications remote from the capture site as an indicators of DNA length. DNA fragments for integrity analysis were amplified from four different loci: 17p13; 5q21; HRMT1L1; LOC91199 (named DIA-D, DIA-E, DIA-X, and DIA-Y, respectively). PCR primer sets and associated TaqMan probe for each locus of interest are “walked” down the chromosome thereby interrogating for the presence and quantitation of increasing length of DNA of approximately, 100 bp, 1300 bp, 1800 bp and 2400 bp fragments of captured DNA. Purified DNA template (5 μl) was mixed with, 5 μl 10×PCR buffer (Takara), 10111 dNTP's (2 mM) (Promega), 0.25111 LATaq (5 U/μl; Takara), 24.751.11 molecular biology grade water (Sigma), 5 μl of a mix of PCR primers (5 μM; Midland) and TaqMan dual-labeled probes (2 μM; Biosearch Technologies). The I-Cycler was programmed as follows: 94° C. for 5 minutes, then 40 cycles of 94° C. for 1 minute, 55° C. for 1 minute, and 72° C. for 1 minute. Genomic standards, prepared as 20, 100, 500, 2500, and 12500 GE/5 μl were prepared and used to generate a standard curve.

### DIA Data Analysis

Threshold Genome Equivalents (GE) values were determined for each of 12 PCR reactions (corresponding to the 1.3 kb, 1.8 kb, and 2.4 kb fragments across the 4 genomic loci) using a previously determined set of cancers and normals. We then applied a requirement that at least 4 of the 12 PCR reactions are above the individual PCR thresholds in order to prospectively determine cancers.

### Statistical Methods

The impact of sample incubation on DNA recovery, and the impact of stabilization buffer on DNA recovery were both assessed using quantitative-PCR analysis. The data for both comparisons were subjected to a paired-sample t-test. The effect of sample incubation on the integrity of recoverable DNA was analyzed using DIA cut-offs. Resulting DIA scores after incubation were analyzed by Fischer's exact test compared to controls that had been analyzed at t0.

### Example 2

### The Effect of Stabilization Buffers on DNA Integrity Assays

The effect of treating a stool sample with a stabilization solution on a subsequent DNA integrity assay (DIA) was examined.

Eight stool samples were removed from a freezer (−80° C.) and thawed. To be included in the study the sample was required to contain at least 90 gm of stool. 30 gm aliquots of each thawed sample was then treated for zero hours, 36 hours without TEN and 36 hours with TEN. 30 gm aliquots of some samples were also treated for 24 hours without TEN and 24 hours with TEN.

The 0 hour samples were homogenized immediately with a 7× excess (i.e., (7 ml/gm) of TEN buffer (10 mM Tris-Cl pH 8.0, 1 mM EDTA and 150 mM NaCl). The remaining samples were allowed to incubate for 24 hours or 36 hours in the presence or absence of TEN buffer.

At the designated times, 210 ml of TEN buffer was added to the samples that did not already have buffer, and the samples were homogenized and frozen at −80° C. The samples were then prepared and processed for DIA analysis essentially as described in US Patent Application Nos. 20040043467 and 20040014104, and in Boynton et al., Clin. Chem. 49:1058-65, 2003. DNA from four genes (named DIA-D, DIA-E, DIA-X, DIA-Y in Table 2) was captured on a solid substrate using a sequence-specific probe. For each gene, interrogation was performed at distances 0.2, 1.3, 1.8, and 2.4 kb from the capture site using real-time polymerase chain reaction (PCR). Positives were identified by determining genomic equivalents (GE) that were above previously determined cutoffs for the four markers.

The results of the analysis are presented in Table 2. Samples and their time points are grouped together. 24 HR NB represents the 24 hour incubation at room temperature without buffer and the 24 HR B represents the 24 hour incubation at room temperature with buffer. The same is true for the 36 hour designations. The 98% specificity cutoff levels for each locus are indicated at the top of Table 2. A DIA marker may be considered positive if the result is above the 98% cutoff level for that marker (e.g., for one of the markers above 200 bp long). A subject may be considered positive if 3 or more (e.g., 4 or more) DIA markers above 200 bp long are positive (even if two or more of the positive DIA markers are at the same locus). The first two samples 30 and 33 are samples that were negative for DIA making it uninformative for comparing the longer DNA markers with and without buffer. However the 200 markers show a stabilizing effect of the addition of buffer all the way through the 36 hour time point.

The next two samples 36 and 32 are those in which the 0 hour is negative for DIA and the samples with buffer at 36 hours are positive. These are probably borderline samples in which there is a slight difference in GE between the 30 gm aliquots causing one to be positive and the other to be negative. However, a stabilizing effect with the addition of buffer can be seen in the 200 markers and in the few longer markers that do have GE numbers.

The next three samples 29, 24 and 38 are those in which the 0 hour, 36 hour and 24 hour (if available) with buffer were all positive and the 36 hour and 24 hour (if available) without buffer were negative. There is a dramatic difference in the GE numbers in both the short 200 markers and the longer markers between those samples with and without buffer once again demonstrating that the addition of buffer appears to be able to stabilize short and long marker GE numbers up through at least 36 hours. The GE numbers for short and long markers in Sample 31 were stable through 36 hours even in the absence of buffer.

The raw GE numbers were subjected to two different comparisons. First, the GE numbers for each of the markers for samples with buffer were compared to the GE numbers for the same samples at 0 hours. The second comparison was between the GE numbers for each marker for the samples with buffer incubated for 24 or 36 hours at room temperature compared to that same samples without buffer incubated for an equivalent time at room temperature. Before carrying out the calculations some of the GE numbers for some of the samples were discarded because they contained a value of 0 (and so could not be used as the value was used as a divisor in subsequent mathematical manipulations), or it appeared that the capture efficiency was lower for that marker than it was relative to the other markers. After discarding the 0's and outliers the following numbers for the average fold increase in GE numbers by marker for the buffer compared to the 0 hour and the buffer compared to the no buffer were derived.

TABLE 3
Average fold increase of samples with buffer compared to 0 hour samples.

| | DIA-D | | | | DIA-E | | | | DIA-X | | | | DIA-Y | | | |
|---|---|---|---|---|---|---|---|---|---|---|---|---|---|---|---|---|
| D100 MuMu | 200 | 1.3 | 1.8 | 2.4 | 200 | 1.3 | 1.8 | 2.4 | 200 | 1.3 | 1.8 | 2.4 | 200 | 1.3 | 1.8 | 2.4 |
| 1.2 | 1.6 | 1.6 | 1.9 | 2.1 | 1.0 | 1.2 | 1.0 | 1.0 | 0.6 | 0.9 | 1.2 | 0.8 | 2.5 | 4.9 | 4.1 | 3.0 |

TABLE 4
Average fold increase of samples with buffer compared to no buffer samples.

| | DIA-D | | | | DIA-E | | | | DIA-X | | | | DIA-Y | | | |
|---|---|---|---|---|---|---|---|---|---|---|---|---|---|---|---|---|
| D100 MuMu | 200 | 1.3 | 1.8 | 2.4 | 200 | 1.3 | 1.8 | 2.4 | 200 | 1.3 | 1.8 | 2.4 | 200 | 1.3 | 1.8 | 2.4 |
| 12.9 | 39.0 | 312.8 | 75.5 | 1.1 | 23.7 | 200.5 | 231.0 | 0.8 | 13.8 | 71.1 | 9.1 | 82.5 | 89.4 | 136.9 | 96.6 | 195.8 |

If the addition of buffer was able to stabilize the GE numbers as efficiently as immediate homogenization then the number would be 1 in the first table above. Numbers less than 1 indicate less stability than immediate homogenization, and numbers greater than 1 show a stabilizing effect greater than immediate homogenization. For the most part the numbers are close to 1, indicating that the addition of buffer prior to incubating up to 36 hours has approximately the same effect as immediate homogenization.

For the table in which the samples with buffer were compared to the samples without buffer, the higher the GE number the greater the stabilizing effect the addition of buffer had. Almost all markers show a dramatic increase in GE numbers with buffer demonstrating that the addition of buffer has a stabilizing effect on the GE numbers for short and long markers. The above data indicate that addition of TEN buffer to stool samples stabilizes the GE numbers for short and long DIA markers in stool samples.

### Example 3

### The Effect of Stabilization Buffers on DNA Integrity and Multiple Mutation Assays

### Effect of Sample Handling Conditions on DIA

All samples were frozen (−80° C.) within an hour of collection from patients in order to arrest any degradation of DNA in the samples. Subsequently samples were thawed, sectioned into aliquots, and further stored under prescribed conditions. All aliquots were then processed to recover and purify human DNA, and the DNA was analyzed using DIA to assess the impact of the sample incubation conditions on the recovered DNA. DIA has been previously described, and shown to detect long DNA fragments in stool as an epigenetic marker for colorectal cancer (Boynton K A, Summerhayes I C, Ahlquist D A, et al. DNA Integrity as a potential marker for stool-based detection of colorectal cancer. Clin. Chem 2003; 49:7 1058-1065). The DIA analysis consists of real-time PCR reactions for 4 independent genetic loci (designated as D, E, X, and Y), and 3 DNA size markers for each locus, corresponding to approximately 1300, 1800, and 2400 base-pair fragments. Therefore every sample of DNA is subjected to 12 independent analyses that yield a direct measure of the copies of DNA of a specified length in the sample of interest. When a threshold value of copies is exceeded (which is specific to each PCR), a marker is determined to be positive. Similarly, if a minimum of 4 of the markers are positive, the sample is considered to be positive for DIA.

An aliquot of every sample was analyzed immediately after thawing (to). A second aliquot from the same sample was stored at room temperature for a prescribed period of time. Stabilization buffer (210 mins) was added to a third aliquot, and stored for the same period of time. Room temperature incubation times were for >36 hours. A total of 38 samples were analyzed by DIA. Of the 38 samples, 11 were found to be DIA positive at to. Table 5 shows the detected copy numbers of each DIA marker for these samples, at to, and extended incubation times, with and without stabilization buffer added. The DIA score (number of positive markers per sample) is also indicated. When no stabilization buffer is added, DNA is significantly degraded in 9 of the 11 stool samples when stored for >36 hours at room temperature. However, for these same samples, addition of stabilization buffer prior to room temperature incubation yields significantly higher DNA copy number, for all of the DIA markers analyzed. As a result, without addition of buffer, samples that originally were DIA-positive (i.e., at to), become DIA-negative, and with addition of stabilization buffer, the samples remain DIA-positive. Two of the samples (GP-031 and GP-079) yielded high quantities of long DNA fragments even upon extended room temperature incubation without any added stabilization buffer.

In addition, for all of the samples where DIA marker degradation was observed, a loss in total human DNA yield was also observed based on real time quantification of a 200 bp human target sequence (Table 5). As observed with the recovery of DIA markers with buffer addition, total human DNA yields were also recovered by addition of buffer to the stool samples. The remaining 27 samples that were not DIA-positive at to were also divided into aliquots, and incubated with and without stabilization buffer. There was no significant amount of long DNA present in these samples in order to judge the effect of incubation conditions on DNA stability. However, by relying on real-time PCR of shorter fragments (e.g., 200 bp) for the same loci (i.e., D, E, X, and Y) the amount of recoverable human DNA was determined. A sampling of results from this group of specimens is shown in Table 6.

TABLE 6
Quantification of recoverable human DNA from selected DIA-negative
samples, incubated with and without stabilization buffer.

| | | D200 | | | |
|---|---|---|---|---|---|
| | Incubation | (GE/ | E200 | X200 | Y200 |
| Sample ID | conditions | 10 ul) | (GE/10 ul) | (GE/10 ul) | (GE/10 ul) |
| GP30 | none | 247 | 196 | 418 | 129 |
| | 36 h no buffer | 50 | 27 | 76 | 0 |
| | 36 h with | 219 | 216 | 390 | 172 |
| | buffer | | | | |
| GP96 | none | 129 | 1120 | 604 | 769 |
| | 36 h no buffer | 63 | 130 | 100 | 100 |
| | 36 h with | 34 | 1270 | 1140 | 1290 |
| | buffer | | | | |
| GP33 | none | 3690 | 13200 | 23300 | 0 |
| | 36 h no buffer | 61 | 136 | 422 | 103 |
| | 36 h with | 2220 | 4640 | 3860 | 2 |
| | buffer | | | | |
| GP125 | none | 21 | 105 | 81 | 74 |
| | 36 h no buffer | 47 | 93 | 68 | 65 |
| | 36 h with | 38 | 68 | 25 | 29 |
| | buffer | | | | |
| LSP49-20 | OHR | 359 | 95 | 91 | 202 |
| | 48 HR NB | 148 | 204 | 101 | 188 |
| | 48 HR B | 310 | 127 | 101 | 411 |
| LSP20-21 | none | 1140 | 807 | 1500 | 337 |
| | 36 h no buffer | 219 | 588 | 421 | 285 |
| | 36 h with | 542 | 363 | 174 | 591 |
| | buffer | | | | |

Similar trends were found for the DIA-negative samples as with the DIA-positive samples. Without any buffer added, the majority of samples (16/27; 59%) stored at room temperature (either 36 or 48 hours) prior to recovery of the DNA, showed significant decrease in human DNA yield, as judged by a direct comparison of the copy number of short fragments compared to the to measurement. The majority of samples (22/27; 81%) were preserved upon addition of stabilization buffer to the stool sample (for example samples GP30 and GP96, in Table 6). In the case of five samples, the addition of stabilization buffer did not offer any significant advantage in recoverable DNA, compared to samples stored without buffer (see for example, LSP20-21 in Table 6).

### Effect of Sample Handling Conditions on Gene Mutation Markers

It has previously been shown that DNA recovered from stool can be interrogated for specific mutations known to be associated with colorectal cancer (CRC) (Ahlquist D A, Skoletsky J E, Boynton K A, et al. Colorectal cancer screening by detection of altered human DNA in stool: Feasibility of a multi-target assay panel. Gastroenterology 2000; 119:1219-1227; Brand R E, Ross M E, Shuber A P. Reproducibility of a Multi-Target Stool-Based Assay for Colorectal Cancer Detection. Am J Gastroenterology (in press); Tagore K S, Lawson M J, Yucaitis J A, et al. Sensitivity and Specificity of a Stool DNA Multi-Target Assay Panel for the Detection of Advanced Colorectal Neoplasia. Clinical Colorectal Cancer 2003; 3 (1), 47-53). In the experiments described above it was shown that upon room temperature incubation of stool samples long fragments of DNA are degraded, significantly diminishing the usefulness of epigenetic DIA markers. Further it was shown that human DNA yield is reduced introducing the question of whether or not sufficient amount of DNA template molecules remain for point mutation analysis in known CRC associated genes (e.g., Kras, APC, and p53). Stool samples from confirmed CRC patients were collected and immediately frozen. All 11 samples were confirmed to contain one or more point mutations, as shown in Table 7.

TABLE 7
Gene mutation analysis results for stool aliquots incubated at room temperature.

| | | DNA | Marker | | | |
|---|---|---|---|---|---|---|
| | | Recovery | (Gene; | SBE | SBE signal | |
| Sample ID | Incubation time | (GE/10 ul) | Condon) | Threshold | 1^{st} pass | 2^{nd} pass |
| GP-003 | to | 2,180 | Kras; k12p2 | 0.400 | 0.445 | 1.290 |
| | 72 h | 351 | | | 1.504 | |
| | to | | p53; 273p1 | 0.200 | 0.999 | 2.419 |
| | 72 h | | | | 1.705 | |
| GP-023 | to | 59,800 | Kras; k12p2 | 0.400 | 1.071 | 1.250 |
| | 72 h | 3,250 | | | 2.423 | |
| GP-024 | to | 509 | BAT-26 | 0.050 | 0.093 | |
| | 48 h | 119 | | | 0.380 | |
| | 72 h | 217 | | | 0.092 | |
| GP-025 | to | 788 | p53; 248p2 | 1.000 | 6.648 | 4.666 |
| | 48 h | 121 | | | 1.206 | |
| | 72 h | 125 | | | 3.697 | |
| GP-026 | to | 458 | p53; 175p2 | 0.400 | 1.459 | 1.459 |
| | 48 h | 160 | | | 1.914 | 1.293 |
| | 72 h | 185 | | | 0.449 | 1.815 |
| GP-034 | to | 72,300 | p53; 248p2 | 1.000 | 1.505 | 1.296 |
| | 36 h | 1,180 | | | 4.834 | |
| GP-029 | to | 10,700 | p53; 175p2 | 0.400 | 1.229 | 1.534 |
| | 36 h | 1,110 | | | 1.721 | 1.641 |
| | 36 h, Buffer | 11,000 | | | 1.793 | 1.459 |
| GP-030 | to | 501 | Kras; k13p2 | 0.400 | 1.814 | 4.203 |
| | 36 h | 135 | | | 3.650 | |
| | 36 h, Buffer | 338 | | | 2.593 | |
| GP-031 | to | 10,300 | APC; 1309 | 0.055 | 0.219 | 0.169 |
| | 36 h | 13,500 | | | 0.090 | |
| | 36 h, Buffer | 4,550 | | | 0.210 | |
| GP-105 | to | 1,540 | APC; 1554 | 0.250 | 1.210 | 1.119 |
| | 48 h | 78 | | | 0.049 | 0.010 |
| | 48 h, Buffer | 9,140 | | | 2.392 | 0.466 |
| GP-121 | to | 4,720 | p53; 175p2 | 0.400 | 2.699 | 3.626 |
| | 48 h | 1,500 | | | 1.601 | 1.181 |
| | 48 h, Buffer | 1,040 | | | 2.329 | 3.628 |

Bold = negative mutation score

Human DNA yield in the sample is judged from the quantifiable recovery of DNA from aliquots stored under the different conditions (Table 7). Without buffer added to the samples, on average only 17% of the DNA in each sample (excluding sample GP-031) was recovered after incubation of samples at room temperature for >36 hours. The one exception, sample GP-031, maintained high DNA yield even without addition of stabilization buffer. Also, as with the DIA experiments, samples incubated with stabilization buffer maintained human DNA yields similar to the tₒ samples.

Aliquots from all samples were analyzed for mutations initially (tₒ), and additional aliquots were analyzed after prescribed room temperature incubation times. Aliquots from the first 6 stool samples (in Table 7) were simply stored at room temperature with no buffer added, whereas aliquots from the next set of 5 samples were stored with and without the addition of stabilization buffer. The SBE signal values listed in Table 7 are derived from a ratio of the CE peak intensity for a mutant specific product, to the peak intensity of an internal control (see Materials and Methods). In this context the values are a crude measure of the percent mutant DNA in the sample (which is typically 1-5%), and must exceed a minimum value (SBE threshold which is specific to each marker) to be judged as positive (i.e., significantly above background). Interestingly, although DNA was degraded for 10 of 11 samples stored at room temperature without buffer added, mutations were reproducibly detected for 9 of the 10, as well as for GP-031 (which did not show degradation of DNA). Sample GP-105, by contrast, was originally shown to contain an APC mutation (at codon 1554), and after incubation of an aliquot for 48 hours without buffer, the mutation was no longer detectable even with repeated analysis. This sample demonstrated a significant reduction in human DNA yield, with only 5% of the original value recovered after room temperature incubation. However, when GP-105 was incubated in the presence of stabilization buffer, the originally identified mutation was detected. In addition, as we observed within the DIA marker experiments, human DNA recovery for all samples remained high when incubated in the presence of stabilization buffer.

### Analysis of Results for DNA Integrity Assays and Gene Mutation Markers

All samples were initially frozen within 1 hour of collection, and then stored frozen until needed. In the first set of experiments, aliquots from 38 samples were analyzed to determine the effect of different sample handling conditions on the DIA marker. Eleven samples were found to be DIA-positive when analyzed immediately after thawing, indicating that long DNA fragments were recovered and amplified from the stool sample. Based on a previously established algorithm (described above), 9 samples went from being DIA-positive to DIA-negative when incubated at room temperature with no buffer added, due to a significant loss of long DNA copies. The loss in long fragments was consistent for each of the loci (D, E, X, and Y) interrogated. In addition, when analyzed for the total yield of human DNA from these samples (i.e., including the shortest fragment size of 200 bp), they all demonstrated a significant loss in recovery. Two of the 11 samples remained DIA-positive even after storing at room temperature >36 hours without stabilization buffer added. When stabilization buffer is added to samples prior to room temperature incubation all of the DIA-positive samples (11/11; 100%) remain positive after >36 hours. We observed some degree of variation in recovery of long fragments. However the variation is not significant with respect to DIA scoring of DNA recovery.

Similar trends were observed relative to the reduction of recoverable human DNA for the 27 samples that were initially DIA-negative. Human DNA recovery was significantly reduced for the majority of samples (25/27; 93%), and only 2 were stable when incubated at room temperature without buffer added. Upon addition of stabilization buffer to stool samples prior to room temperature incubation, human DNA yield was maintained in 81% (22/27) of the samples. There was no apparent benefit upon addition of buffer for 5 samples, whereas with DIA-positive samples all (11/11) benefited, in terms of DNA integrity or human DNA yield. This difference is likely linked to the fact that the DIA-negative samples contained less recoverable human DNA overall. A DNA recovery value for each sample was calculated by averaging the recovery values for all 4 loci of the 200 bp PCR for each sample. The median recovery for the DIA-negative samples was 452 genome equivalents (GE), and 8,410 for the DIA-positive samples (P<0.0001). Overall, this suggests that DIA-positive samples have almost 20-fold more human DNA recoverable from stool. Without addition of stabilization buffer these samples might otherwise become false-negatives under adverse incubation conditions.

Results of the matched aliquots with and without stabilization buffer were first subjected to statistical analysis without any assumptions as to the mechanism of action with the buffer. Using a one-tailed paired t-test, we found a significant decrease (P=0.0018) in recovered DNA, without addition of buffer, for aliquots that had been incubated at room temperature (control=to). The analysis was based on quantitative PCR data for the shortest DNA fragment size (i.e., 200 bp) for all samples. Likewise the addition of buffer to samples prior to room temperature incubation was found to yield a significant increase in DNA recovery, relative to the matched aliquots with no buffer added (P=0.010). Finally, in order to determine the significance of the addition of stabilization buffer on DIA scores (and therefore the integrity of the DNA), results were subjected to a Fischer's exact test, analyzing the samples that were found to be DIA positive at to (N=11). Addition of buffer significantly preserves DNA integrity (P=0.0002) compared to aliquots with no buffer addition. Therefore, whereas 9 of 11 samples became DIA-negative after incubation at room temperature when no buffer was added, buffer addition led to a recovery of the 9 samples, such that even after 36 h incubation all of the samples (11 of 11) were positive.

In order to determine the effect of DNA degradation on stool DNA mutation detection, analyses were performed on stool samples that were informative for specific DNA associated point mutations. In the second set of experiments, 11 samples were determined to be positive by at least one point mutation marker in a mutation panel, and the samples were then re-analyzed with the mutation panel after sample aliquots had been incubated under the same incubation conditions as described above. Without addition of stabilization buffer, the recoverable DNA in 10 of 11 samples was significantly diminished. On average, for these samples, 83% of the original DNA was lost. Only one sample did not demonstrate a loss in human DNA yield upon room temperature incubation. Despite the loss of DNA, the original mutations could be confirmed after room temperature incubation in 10 of 11 samples (91%). As an example, one sample had 2180 GE of recoverable human DNA at to, and was originally found to be positive for a Kras and a p53 marker. Upon room temperature incubation of the stool aliquot for 72 hours, only 351 GE of amplifiable DNA was recovered, yet the sample remained positive for both markers. One sample, in addition to having a significant reduction in human DNA yield, also had a loss of an APC mutation originally detected at to. However, with stabilization buffer added, a high yield of human DNA was measured after 48 hrs of incubation, and the sample remained positive for the APC mutation.

Results from these experiments indicate that DIA is quite sensitive to sample incubation conditions, and DNA degradation, whereas the mutation panel is more refractory. In one aspect of the invention, incubation of samples at room temperature for 36 hours or more in the absence of stabilization buffer may degrade DNA to the point that any remaining long fragments fall below an DNA integrity assay cut-off. A point mutation assay, on the other hand, is dependent on the detection of mutant DNA in the presence of wild-type, where the percent mutant is often only on the order of 1% of the total human DNA, and PCR amplification efficiency is not dependent on the size of the template molecules. Therefore even in the case of adverse incubation conditions, as long as at least a single copy of mutant sequence is recovered and amplified in the PCR reactions, it should be possible to detect mutations downstream in the analytical process. Interestingly, of the mutation positive samples that had been stored without stabilization buffer, the one where the mutation was lost had the lowest measurable DNA, suggesting that the lack of detection was likely due to an insufficient number of mutant molecules to yield a signal above background. In addition, even though the remaining mutation positive samples remained positive with or without buffer, a consistent loss of human DNA yield was observed in all samples when stabilization buffer was not added.

According to aspects of the invention, despite the stability of a point mutation analysis, it may be desirable to mitigate degradation of DNA, particularly when the amount of abnormal DNA in a sample may be very low. According to the invention, in order to maximize clinical sensitivity of fecal DNA assays it is important to maximize the recovery of otherwise scarce amounts of human DNA. Accordingly, aspects of the invention may be particularly useful for detecting indicia of adenoma and/or early stage cancer. In one embodiment, the invention may be useful to avoid or reduce the number of false negative results in a screen of a population of individuals for one or more indicia of adenoma or early stage cancer (e.g., using a DNA integrity assay, a multiple mutation assay, a hypermethylation assay, or any combination thereof).

In summary, under adverse sample handling conditions, 91% of samples (10/11; 95% CI: 59-100%) that were positive by the point mutation panel remain positive. Under the same conditions, however, only 18% of samples (2/11; 95% CI: 2-52%) that were DIA-positive when analyzed fresh, remained positive after room temperature incubation. Transporting samples frozen is a viable means of temporarily neutralizing active nucleases and mitigating degradation of DNA. However, insuring that samples are frozen immediately, and keeping samples frozen during transport to test-facilities poses challenges and risks in a population based screening application of fecal DNA assays. According to the invention, an alternative is to add a stabilization buffer to the sample as a way of preserving DNA such that samples could be stored (or transported) at room temperature. The results of these experiments show that all DIA-positive samples (11/11; 100%), and all mutation panel positive samples (11/11; 100%) remained positive after storing for >36 hours after addition of stabilization buffer to the stool.

In any of the aspects of the invention described herein, including the examples, a sample (e.g., a stool sample) may be directly deposited into a container (e.g., a sealable container) that already contains stabilization solution. The amount of solution may be sufficient to cover the sample or a portion thereof. Alternatively, stabilization solution may be added to a container when (e.g., at the same time, immediately after, or within minutes, hours, or a day) a biological sample (e.g., a stool sample) is deposited in the container. The amount of solution added may be sufficient to cover the entire sample or at least a portion thereof (e.g., 10%, 25%, 50%, or more). In one embodiment, an amount of solution sufficient to fill the container may be added. In some embodiment, a container may be between about 25 mls and about 1,000 mls (e.g., between about 50 mls and about 500 mls, between about 100 mls and about 400 mls, or about 250 mls). However, smaller or larger containers may be used. A container with sample and stabilization solution may be sealed for storage/shipping.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for preparing a nucleic acid for analysis, the method comprising:
providing a patient sample comprising a nucleic acid and shed cells or cellular debris; and
contacting said patient sample with a stabilization solution under conditions sufficient to stabilize said nucleic acid for nucleic acid integrity analysis and/or multiple mutation analysis, wherein said stabilization solution comprises a buffer, a salt, and a chelating agent, thereby preparing a nucleic acid for a nucleic acid integrity analysis.

2. The method of claim 1, wherein the conditions are sufficient to detect at least a three-fold genomic equivalent (GE) increase in a nucleic acid integrity analysis of a patient sample having adenoma or cancer as compared to the GE detected in a nucleic acid integrity analysis of a sample from said patient that is not incubated with said stabilization solution.

3. The method of claim 1, wherein the integrity analysis is performed by determining an amount of nucleic acid greater than about 200 nucleotides in length using an assay that detects wild-type or mutant nucleic acid, wherein said nucleic acid is present in a patient sample comprising shed cells or cellular debris; and identifying said patient as having cancer or adenoma if said amount is greater than an amount of nucleic acid expected to be present in a sample obtained from a patient who does not have cancer or adenoma.

4. The method of claim 1, wherein the patient sample is a sample from a human.

5. The method of claim 1, wherein the patient sample is a stool sample.

6. The method of claim 3, wherein the patient sample is a stool sample.

7. The method of claim 1, wherein the patient sample is frozen and thawed prior to incubation with stabilization solution.

8. The method of claim 1, wherein the patient sample is not frozen prior to incubation with said stabilization solution.

9. The method of claim 1, wherein the stabilization solution is present at 1-7 ml per gram of patient sample.

10. The method of claim 1, wherein the patient sample and stabilization solution are incubated at about 17 to 28 degrees Centigrade.

11. The method of claim 1, wherein the patient sample and stabilization solution are incubated at about 20 to 25 degrees Centigrade.

12. The method of claim 1, wherein the patient sample and stabilization solution are incubated at least 6 hours, at least 12 hours, at least 24 hours, or at least 36 hours.

13.-15. (canceled)

16. The method of claim 1, wherein the buffer in said stabilization solution is 0.5 mM to 25 mM Tris, 5 mM to 15 mM Tris, 8 mM to 13 mM Tris. or 10 mM Tris.

17.-19. (canceled)

20. The method of claim 1, wherein the chelating agent in said stabilization solution is 0.01 to 2.5 mM EDTA, 0.75-1.25 mM EDTA, or 1 mM EDTA.

21.-22. (canceled)

23. The method of claim 1, wherein the salt in said stabilization solution is 75 mM to 225 mM NaCl, 100 mM to 175 mM NaCl, or 150 mM NaCl.

24.-25. (canceled)

26. The method of claim 1, wherein said stabilization solution is provided at pH 7.0 to 9.0, pH 7.5 to 8.5, or about pH 8.0.

27.-28. (canceled)

29. The method of claim 1, further comprising:
determining in said incubated patient sample an amount of nucleic acid greater than about 200 bp in length using an assay that detects wild-type or mutant nucleic acid; and identifying said patient as having cancer or adenoma if said amount is greater than an amount of nucleic acid expected to be present in a sample obtained from a patient who does not have cancer or adenoma.

30. A method for preparing a nucleic acid for an assay for diagnosing a carcinoma or adenoma, the method comprising:
providing a patient stool sample comprising shed cells or cellular debris and a nucleic acid; and
contacting said patient sample with a stabilization solution under conditions sufficient to stabilize said nucleic acid for nucleic acid integrity and/or multiple mutation analysis, wherein said stabilization solution is about pH 7.5 to about pH 8.5 and comprises 0.5 mM to 25 mM Tris, 0.01 to 2.5 mM EDTA and 100 mM to 200 mM NaCl, and wherein the conditions are sufficient to detect at least a three-fold genomic equivalent (GE) increase in a nucleic acid integrity analysis of a patient sample having adenoma or cancer as compared to the GE detected in a nucleic acid integrity analysis of a sample from said patient that is not incubated with said stabilization solution, thereby preparing a nucleic acid for a nucleic acid integrity analysis.
